(19) United States
(12) Patent Application Publication
(10) Pub. No.: US 2018/0140279 A1
(43) Pub. Date: May 24, 2018

(54) METHOD AND SYSTEM FOR ENHANCED DETECTION AND VISUALIZATION OF A SURGICAL NEEDLE IN ULTRASOUND DATA BY PERFORMING SHEAR WAVE ELASTICITY IMAGING

(71) Applicant: General Electric Company, Schenectady, NY (US)

(72) Inventor: Christian Fritz Perrey, Zipf (AT)

(21) Appl. No.: 15/358,467

(22) Filed: Nov. 22, 2016

(51) Int. Cl. A61B 8/08; A61B 8/00; A61B 10/02

(52) U.S. Cl. CPC A61B 8/485; A61B 8/0841; A61B 10/0233; A61B 8/461; A61B 8/5207

(57) ABSTRACT

An ultrasound system provides enhanced detection and visualization of a surgical needle in ultrasound data by performing shear wave elasticity imaging. A shear wave is induced in biological tissue having a surgical needle inserted therein. The ultrasound system acquires shear wave ultrasound data at a high pulse repetition frequency from the biological tissue having the surgical needle. A processor of the ultrasound system processes the shear wave ultrasound data to generate a map of the biological tissue and the surgical needle. A display system of the ultrasound system displays a representation of the surgical needle overlaid on an ultrasound image.

### CROSS-REFERENCE TO RELATED APPLICATIONS/INCORPORATION BY REFERENCE

[Not Applicable]

### FIELD

Certain embodiments relate to ultrasound imaging. More specifically, certain embodiments relate to a method and system for enhanced detection and visualization of a surgical needle in ultrasound data by performing shear wave elasticity imaging.

### BACKGROUND

Ultrasound imaging is a medical imaging technique for imaging organs and soft tissues in a human body. Ultrasound imaging uses real time, non-invasive high frequency sound waves to produce a two-dimensional (2D) image and/or a three-dimensional (3D) image.

Elastography is a medical imaging modality that maps the elastic properties of soft tissue. It can be useful in medical diagnoses because it can discern healthy from unhealthy tissue for specific organs and/or growths. For example, malignant tumors will often be harder than the surrounding tissue, and diseased livers are stiffer than healthy ones. Elastography has been used to guide or replace biopsies, for example, by identifying potentially cancerous tissue or other diseased tissue based on tissue stiffness.

Several techniques are known for performing ultrasound elastography. Compression-based elastography is performed by applying an external compression to tissue and comparing ultrasound images before compression and during compression. Spectral tracking techniques may be used to track tissue deformation. The areas of the image that are least deformed have a higher stiffness, while the most deformed areas have the least stiffness. Another ultrasound elastography technique includes shear wave elasticity imaging. In shear wave elasticity imaging, a push disturbance is induced in the tissue by the force of a focused ultrasound beam or by an external push, for example. The push disturbance generates shear waves that propagate laterally from the point of disturbance. The ultrasound device acquires image data of the shear waves and determines how fast the shear waves travel through different lateral positions within the tissue. An elasticity map may be created based on the shear wave speed.

Ultrasound imaging may be useful in positioning an instrument at a desired location inside a human body. For example, in order to perform a biopsy on a tissue sample, it is important to accurately position a biopsy needle so that the tip of the biopsy needle penetrates the tissue to be sampled. By viewing the biopsy needle using an ultrasound imaging system, the biopsy needle can be directed toward the target tissue and inserted to the required depth. Thus, by visualizing both the tissue to be sampled and the penetrating instrument, accurate placement of the instrument relative to the tissue can be achieved.

A needle is a specular reflector, meaning that it behaves like a mirror with regard to the ultrasound waves reflected off of it. The ultrasound is reflected away from the needle at an angle equal to the angle between the transmitted ultrasound beam and the needle. Ideally, an incident ultrasound beam would be substantially perpendicular with respect to a surgical needle in order to visualize the needle most effectively. The smaller the angle at which the needle is inserted relative to the axis of the transducer array, i.e., the imaginary line normal to the face of the transducer array, the more difficult it becomes to visualize the needle. In a typical biopsy procedure using a linear probe, the geometry is such that most of the transmitted ultrasound energy is reflected away from the transducer array face and thus is poorly detected by the ultrasound imaging system.

Several proposals have been made for improving ultrasound visualization of surgical needles. For example, a rough coating may be applied to a length of a needle shaft to provide increased acoustical scattering that may provide brighter ultrasound images of the needle. However, the use of coatings can be expensive and may require specialized needles. As another example, image processing techniques may be used to attempt to track a position of a needle to assist with predicting and identifying subsequent needle positions. However, needle tracking imaging processing techniques may be complex and otherwise technically difficult to implement. Compression-based elastography has also been proposed as a possible technique for determining a needle position. For example, the insertion or other movement of the needle may deform surrounding tissue such that ultrasound images before insertion/movement and during insertion/movement may be compared to identify the tissue deformation and locate the needle position. However, moving a needle or otherwise compressing tissue to identify a needle position may not be desirable when a needle is near a tumor. More specifically, moving a needle to determine the needle position may result in misplacement of the needle.

Further limitations and disadvantages of conventional and traditional approaches will become apparent to one of skill in the art, through comparison of such systems with some aspects of the present invention as set forth in the remainder of the present application with reference to the drawings.

### BRIEF SUMMARY

A system and/or method is provided for enhanced detection and visualization of a surgical needle in ultrasound data by performing shear wave elasticity imaging, substantially as shown in and/or described in connection with at least one of the figures, as set forth more completely in the claims.

These and other advantages, aspects and novel features of the present invention, as well as details of an illustrated embodiment thereof, will be more fully understood from the following description and drawings.

### BRIEF DESCRIPTION OF SEVERAL VIEWS OF THE DRAWINGS

FIG. 1 is a block diagram of an exemplary ultrasound system that is operable to provide enhanced detection and visualization of a surgical needle in ultrasound data by performing shear wave elasticity imaging, in accordance with various embodiments.

FIG. 2 is a flow chart illustrating exemplary steps that may be utilized for providing enhanced detection and visualization of a surgical needle in ultrasound data by performing shear wave elasticity imaging, in accordance with various embodiments.

### DETAILED DESCRIPTION

Certain embodiments may be found in a method and system for enhanced detection and visualization of a surgical needle in ultrasound data by performing shear wave elasticity imaging. Various embodiments provide the technical effect of providing a system configured to generate shear waves in biological tissue, acquire ultrasound image data of the generated shear waves, process the ultrasound image data to determine a local distribution of shear wave speed, and convert the local distribution of the shear wave speed to a map that is used to identify a location of a surgical needle in the tissue.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or a block of random access memory, hard disk, or the like) or multiple pieces of hardware. Similarly, the programs may be stand alone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized and that structural, logical and electrical changes may be made without departing from the scope of the various embodiments. The following detailed description is, therefore, not to be taken in a limiting sense, and the scope of the present invention is defined by the appended claims and their equivalents.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

Also as used herein, the term “image” broadly refers to both viewable images and data representing a viewable image. However, many embodiments generate (or are configured to generate) at least one viewable image. In addition, as used herein, the phrase “image” is used to refer to an ultrasound mode such as B-mode, CF-mode, and/or sub-modes of B-mode and/or CF such as Shear Wave Elasticity Imaging (SWEI), TVI, Angio, B-flow, BMI, BMI_Angio, and in some cases also MM, CM, PW, TVD, CW where the “image” and/or “plane” includes a single beam or multiple beams.

Furthermore, the term processor or processing unit, as used herein, refers to any type of processing unit that can carry out the required calculations needed for the various embodiments, such as single or multi-core: CPU, Graphics Board, DSP, FPGA, ASIC or a combination thereof.

It should be noted that various embodiments described herein that generate or form images may include processing for forming images that in some embodiments includes beamforming and in other embodiments does not include beamforming. For example, an image can be formed without beamforming, such as by multiplying the matrix of demodulated data by a matrix of coefficients so that the product is the image, and wherein the process does not form any “beams”. Also, forming of images may be performed using channel combinations that may originate from more than one transmit event (e.g., synthetic aperture techniques).

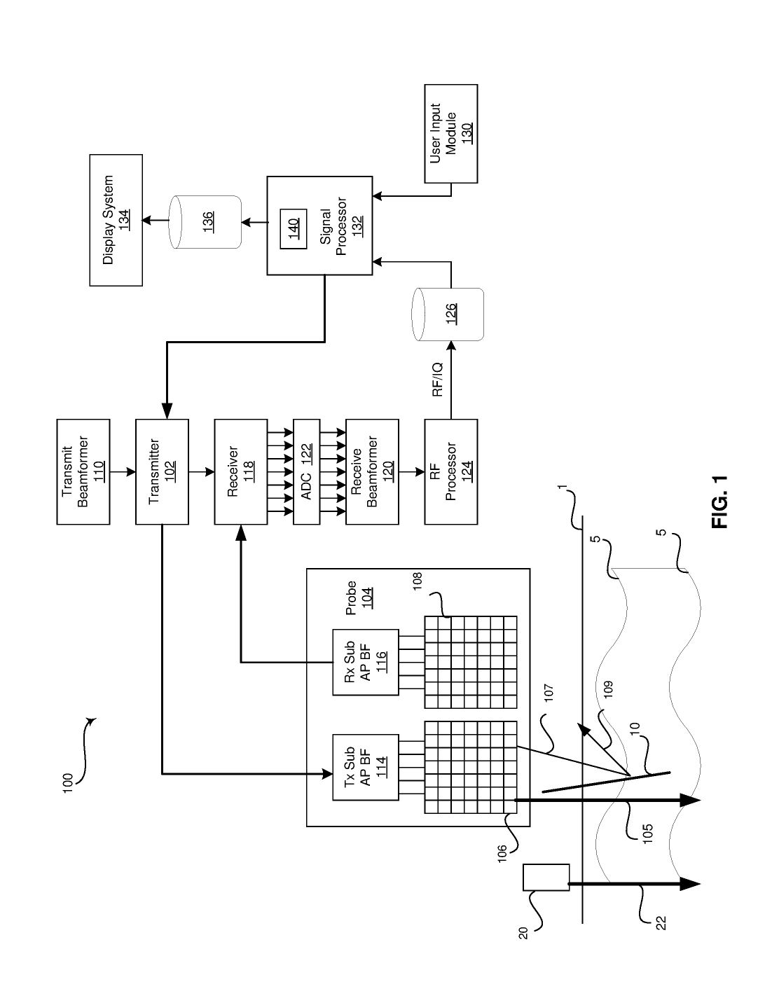

In various embodiments, ultrasound processing to form images is performed, for example, including ultrasound beamforming, such as receive beamforming, in software, firmware, hardware, or a combination thereof. One implementation of an ultrasound system having a software beamformer architecture formed in accordance with various embodiments is illustrated in FIG. 1.

FIG. 1 is a block diagram of an exemplary ultrasound system 100 that is operable to provide enhanced detection and visualization of a surgical needle 10 in ultrasound data by performing shear wave elasticity imaging, in accordance with various embodiments. Referring to FIG. 1, there is shown a surgical needle 10, an ultrasound system 100, and an external vibration device 20. The surgical needle 10 comprises a distal insertion end and a proximal hub end. The proximal hub end may be grasped by an operator of the needle 10 and the distal insertion end may be inserted into biological tissue 1 to, for example, perform an ultrasound guided biopsy or any suitable procedure. The external vibration device 20 may be configured to provide an external push force 22 for displacing tissue to create shear waves 5 in the tissue 1. Additionally and/or alternatively, the ultrasound system 100 may provide the push force, such as a high intensity ultrasound push pulse 105, for generating the shear waves 5 in the tissue 1. The ultrasound system 100 comprises a transmitter 102, an ultrasound probe 104, a transmit beamformer 110, a receiver 118, a receive beamformer 120, a RF processor 124, a RF/IQ buffer 126, a user input module 130 a signal processor 132, an image buffer 136, and a display system 134.

The transmitter 102 may comprise suitable logic, circuitry, interfaces and/or code that may be operable to drive an ultrasound probe 104. The ultrasound probe 104 may comprise a one dimensional (1D, 1.25D, 1.5D or 1.75D) array or two dimensional (2D) array of piezoelectric elements. The ultrasound probe 104 may comprise a group of transmit transducer elements 106 and a group of receive transducer elements 108, that normally constitute the same elements.

The transmit beamformer 110 may comprise suitable logic, circuitry, interfaces and/or code that may be operable to control the transmitter 102 which, through a transmit sub-aperture beamformer 114, drives the group of transmit transducer elements 106 to emit high intensity ultrasound push pulses 105 into a point of disturbance of the tissue 1 and to emit ultrasonic transmit signals 107 into a region of interest of the tissue 1. As used herein, the term “high intensity ultrasound push pulses” refers to a derated spatial-peak temporal-average intensity (I_{SPTA.3}) of between 200 and 700 mW/cm^{2.} The transmitted high intensity ultrasound push pulses 105 may displace the tissue 1 to create shear waves 5 propagating laterally from the point of disturbance. The transmitted ultrasonic signals 107 may be back-scattered from structures in the object of interest, like the tissue 1 as deformed by the shear waves 5, and surgical instruments in the object of interest, like a surgical needle 10, to produce echoes 109. The echoes 109 are received by the receive transducer elements 108. The group of receive transducer elements 108 in the ultrasound probe 104 may be operable to convert the received echoes into analog signals, undergo sub-aperture beamforming by a receive sub-aperture beamformer 116 and are then communicated to a receiver 118.

The receiver 118 may comprise suitable logic, circuitry, interfaces and/or code that may be operable to receive and demodulate the signals from the receive sub-aperture beamformer 116. The demodulated analog signals may be communicated to one or more of the plurality of A/D converters 122. The plurality of A/D converters 122 may comprise suitable logic, circuitry, interfaces and/or code that may be operable to convert the demodulated analog signals from the receiver 118 to corresponding digital signals. The plurality of A/D converters 122 are disposed between the receiver 118 and the receive beamformer 120. Notwithstanding, the invention is not limited in this regard. Accordingly, in some embodiments, the plurality of A/D converters 122 may be integrated within the receiver 118.

The receive beamformer 120 may comprise suitable logic, circuitry, interfaces and/or code that may be operable to perform digital beamforming processing on the signals received from the plurality of A/D converters 122. The resulting processed information may be converted back to corresponding RF signals. The corresponding output RF signals that are output from the receive beamformer 120 may be communicated to the RF processor 124. In accordance with some embodiments, the receiver 118, the plurality of A/D converters 122, and the beamformer 120 may be integrated into a single beamformer, which may be digital.

The RF processor 124 may comprise suitable logic, circuitry, interfaces and/or code that may be operable to demodulate the RF signals. In accordance with an embodiment, the RF processor 124 may comprise a complex demodulator (not shown) that is operable to demodulate the RF signals to form I/Q data pairs that are representative of the corresponding echo signals. The RF or I/Q signal data may then be communicated to an RF/IQ buffer 126.

The RF/IQ buffer 126 may comprise suitable logic, circuitry, interfaces and/or code that may be operable to provide temporary storage of the RF or I/Q signal data, which is generated by the RF processor 124.

The user input module 130 may be utilized to initiate shear wave elasticity imaging, change scan mode, input patient data, surgical instrument data, scan parameters, settings, configuration parameters, and the like. In an exemplary embodiment, the user input module 130 may be operable to configure, manage and/or control operation of one or more components and/or modules in the ultrasound system 100. In this regard, the user input module 130 may be operable to configure, manage and/or control operation of transmitter 102, the ultrasound probe 104, the transmit beamformer 110, the receiver 118, the receive beamformer 120, the RF processor 124, the RF/IQ buffer 126, the user input module 130, the signal processor 132, the image buffer 136, and/or the display system 134. The user input module 130 may be located at various positions on and/or around the ultrasound system 100 such as on the probe 104, at a control panel, and/or at any suitable location.

The signal processor 132 may comprise suitable logic, circuitry, interfaces and/or code that may be operable to process ultrasound information (i.e., RF signal data or IQ data pairs) for presentation on a display system 134. The signal processor 132 is operable to perform one or more processing operations to determine the position and orientation information of a surgical needle 10. The signal processor 132 is operable to perform one or more processing operations according to a plurality of selectable ultrasound modalities on the acquired ultrasound information. Acquired ultrasound information may be processed in real-time during a scanning session as the echo signals are received. Additionally or alternatively, the ultrasound information may be stored temporarily in the RF/IQ buffer 126 during a scanning session and processed in less than real-time in a live or off-line operation. In the exemplary embodiment, the signal processor 132 may comprise a shear wave elastography processing module 140.

The ultrasound system 100 may be operable to continuously acquire ultrasound information at a frame rate that is suitable for the imaging situation in question. Typical frame rates range from 20-70 but may be lower or higher. For example, shear wave elasticity imaging may have higher frame rates related to the high pulse repetition frequency used to image shear waves 5 in tissue 1. In various embodiments, the pulse repetition frequency in a shear wave elasticity imaging mode is at least 300 pulses/second, and preferably greater or equal to 1000 pulses/second. The acquired ultrasound information may be displayed on the display system 134 at a display-rate that can be the same as the frame rate, or slower or faster. An image buffer 136 is included for storing processed frames of acquired ultrasound information that are not scheduled to be displayed immediately. Preferably, the image buffer 136 is of sufficient capacity to store at least several seconds worth of frames of ultrasound information. The frames of ultrasound information are stored in a manner to facilitate retrieval thereof according to its order or time of acquisition. The image buffer 136 may be embodied as any known data storage medium.

The shear wave elastography processing module 140 may comprise suitable logic, circuitry, interfaces and/or code that may be operable to handle processing of shear wave ultrasound data to provide enhanced detection and visualization of a surgical needle by creating a map that represents a distribution of the shear wave speed, elasticity, and/or the spatial gradient. As used herein, the term “shear wave ultrasound data” refers to ultrasound information received at the signal processor 132 corresponding with the received echoes 109 produced by the back-scattering of the transmitted ultrasonic signals 107 from structures (e.g., tissue 1) and surgical instruments (e.g., surgical needle 10) in the object of interest as deformed by the shear waves 5. In this regard, the shear wave elastography processing module 140 may comprise suitable logic, circuitry, interfaces and/or code that may be operable to handle processing the shear wave ultrasound data to determine a local distribution of shear wave speed in the tissue 1. The shear wave speed may be computed by direct inversion of the Helmholtz equation, time-of-flight measurement, or any suitable computational method. The shear wave ultrasound data may be acquired after a push disturbance is induced in the tissue 1 by the force of a focused ultrasound beam 105 or by an external push force 22, for example. The push disturbance 105, 22 generates shear waves 5 that propagate laterally from the point of disturbance. The ultrasound system 100 acquires the shear wave ultrasound data using a high pulse repetition frequency. As used herein, the term “high pulse repetition frequency” refers to a pulse repletion frequency of at least 300 pulses/second. In a preferred embodiment, the pulse repetition frequency used to acquire shear wave ultrasound data is greater or equal to 1000 pulses/second.

The shear wave elastography processing module 140 may comprise suitable logic, circuitry, interfaces and/or code that may be operable to convert the local distribution of shear wave speed in the tissue 1 to a map, such as a velocity distribution map, an elasticity map, a spatial gradient map, or any suitable map representing the contrast between the needle 10 and surrounding tissue 1. For example, the local distribution may be mapped based on the shear wave speed to generate a velocity distribution map. As another example, the local distribution may be converted to the elasticity map by computing the stiffness based on Young's modulus, a similar shear modulus, or any suitable conversion computation. Moreover, in various embodiments, a spatial gradient filter may be applied to the velocity distribution map and/or elasticity map to generate a spatial gradient map providing enhanced visualization of the needle 10.

The map represents the speed that the shear wave passed through the tissue at lateral locations from the point of disturbance in the shear wave ultrasound data. The shear wave propagation velocity corresponds to the stiffness of the tissue at the lateral locations. Specifically, the higher shear wave velocity corresponds with more stiffness and the lower shear wave velocity corresponds with less stiffness. Based on the difference in velocity and/or elasticity of the needle 10 and surrounding tissue 10 at the lateral locations and the size and shape of the needle 10, the position of the surgical needle 10 is readily identifiable. For example, the maps may be color-coded or grayscale maps having a range of colors or grays that correspond with the shear wave speed and/or elasticity. Specifically, an elasticity map may have dark blue or dark gray/black corresponding with soft elasticity to red or light gray/white corresponding with hard elasticity, among other things. The surgical needle 10 is a characteristic shape and has a stiffer elasticity than tissue 1 so that a representation of the needle 10 may appear in the elasticity map as a thin straight red or light gray/white line. The map having the representation of the surgical needle 10 may be displayed on the display system 134. For example, the map having the representation of the surgical needle 10 may be overlaid on an ultrasound image such as a B-mode image or any suitable ultrasound image.

Additionally and/or alternatively, the shear wave elastography processing module 140 may comprise suitable logic, circuitry, interfaces and/or code that may be operable to perform image segmentation on the surgical needle 10 within the map. In various embodiments, the shear wave elastography processing module 140 may perform the image segmentation semi-automatically or automatically. For example, semi-automatic image segmentation may be performed by an operator identifying the region of interest having the surgical needle 10 and the shear wave elastography processing module 140 applies image processing algorithms to segment the surgical needle 10 within the region of interest. As another example, automatic image segmentation may be performed by the shear wave elastography processing module 140 applying image processing algorithms to segment the surgical needle 10 without operator input. The image processing algorithms may be based at least in part on needle detection parameters such as color or grayscale information corresponding with needle elasticity, among other things. A representation of the needle shape corresponding with the segmented surgical needle 10 may be overlaid on the B-mode image or any suitable ultrasound image to enhance the visualization of the surgical needle 10.

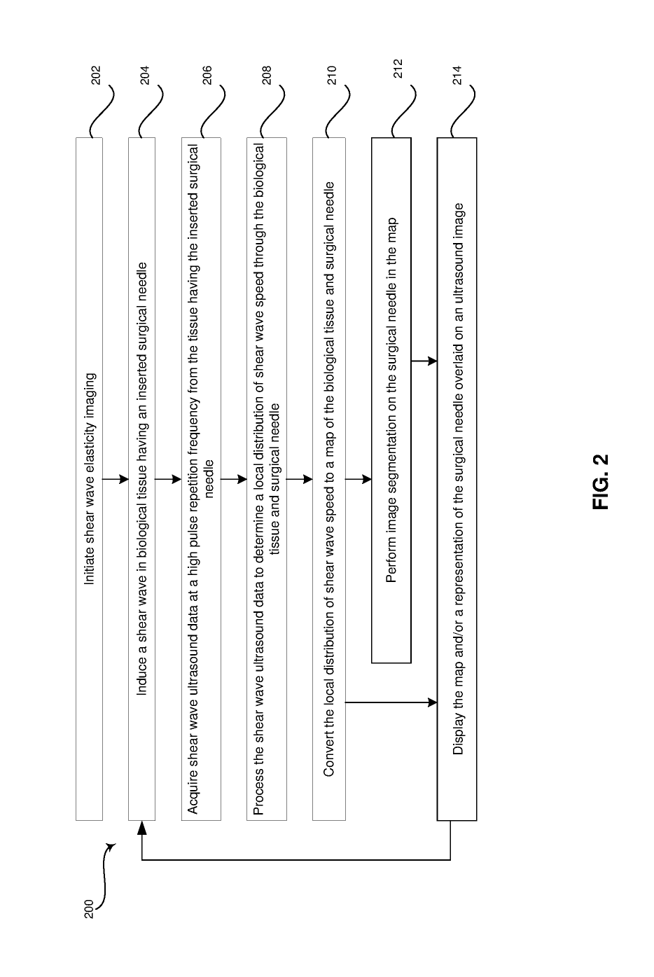

FIG. 2 is a flow chart illustrating exemplary steps that may be utilized for providing enhanced detection and visualization of a surgical needle 10 in ultrasound data by performing shear wave elasticity imaging, in accordance with various embodiments. Referring to FIG. 2, there is shown a flow chart 200 comprising exemplary steps 202 through 214. Certain embodiments may omit one or more of the steps, and/or perform the steps in a different order than the order listed, and/or combine certain of the steps discussed below. For example, some steps may not be performed in certain embodiments. As a further example, certain steps may be performed in a different temporal order, including simultaneously, than listed below.

In step 202, the signal processor 132 in the ultrasound system 100 may be operable to initiate shear wave elasticity imaging. For example, the signal processor 132 may receive an input from user input module 130 providing instructions for performing shear wave elasticity imaging. The instructions may correspond with a one-time request for performing shear wave elasticity imaging or a request for continuous or periodic performance of shear wave elasticity imaging. As an example, an operator performing an ultrasound guided biopsy may not be able to identify a surgical needle 10 in a B-mode ultrasound image and may press a button 130 at an ultrasound probe 104 or control panel for initiating shear wave elasticity imaging to assist with the detection and visualization of the surgical needle 10. As another example, the operator may input instructions at the user input module 130 for continuously or periodically performing shear wave elasticity imaging at the beginning or during an ultrasound guided biopsy or any suitable procedure. Steps 204 through 214 may be repeated continuously and/or periodically based on the operator input instructions.

In step 204, shear waves 5 are induced in biological tissue 1 having an inserted surgical needle 10. For example, an ultrasound probe 104 of the ultrasound system 100 may provide a push force, such as a high intensity ultrasound push pulse 105, for generating the shear waves 5 in the tissue 1. As another example, an external vibration device 20 may be configured to provide an external push force 22 for displacing tissue to create shear waves 5 in the tissue 1.

In step 206, shear wave ultrasound data is acquired by the ultrasound system 100 at a high pulse repetition frequency from the tissue having the inserted surgical needle. The shear wave ultrasound data is in a region that includes biological tissue 1 and the surgical needle 10. The pulse repetition frequency in the shear wave elasticity imaging mode is at least 300 pulses/second, and preferably greater or equal to 1000 pulses/second.

In step 208, the signal processor 132 may process the shear wave ultrasound data to determine a local distribution of shear wave speed through the biological tissue 1 and surgical needle 10. For example, the shear wave elastography processing module 140 of the signal processor 132 may compute the shear wave speed at locations in the shear wave ultrasound data by direct inversion of the Helmholtz equation, time-of-flight measurement, or any suitable computational method.

In step 210, the signal processor 132 may convert the local distribution of shear wave speed through the biological tissue and surgical needle to a map. In various embodiments, the map may be a velocity distribution map, an elasticity map, a spatial gradient map, or any suitable map representing the contrast between the needle 10 and surrounding tissue 1. For example, the shear wave elastography processing module 140 of the signal processor 132 may map the local distribution based on the shear wave speed to generate a velocity distribution map. As another example, the shear wave elastography processing module 140 may convert the local distribution to an elasticity map by computing the stiffness based on Young's modulus, a similar shear modulus, or any suitable conversion computation. Moreover, in various embodiments, the shear wave elastography processing module 140 may apply a spatial gradient filter to the velocity distribution map and/or elasticity map to generate a spatial gradient map. The map may be a color-coded or grayscale map having different colors or shades of gray corresponding to different velocities and/or elasticities. For example, a color-coded or grayscale elasticity map may show soft tissue 1 as a dark blue or dark gray/black, whereas the needle 10 having a greater stiffness than tissue 1 can be shown in red or light gray/white, among other things.

In step 212, the signal processor 132 may perform image segmentation on the surgical needle 10 in the map. For example, the shear wave elastography processing module 140 of the signal processor 132 may apply image processing algorithms to segment the surgical needle 10. The image processing algorithms may include parameters, such as color or grayscale information associated with particular elasticities, applied to segment the needle 10. The image segmentation may be performed semi-automatically such as with the assistance of an operator identifying the region of interest having the surgical needle 10. Additionally and/or alternatively, the image segmentation may be performed automatically by applying the image processing algorithms without operator input.

In step 214, the map and/or a representation of the surgical needle 10 may be overlaid on an ultrasound image. For example, the map having a representation of the surgical needle 10 from step 210 may be superimposed on the ultrasound image and presented at display system 134. As another example, the segmented needle 10 from step 212 may be superimposed on the ultrasound image and presented at the display system 134. The ultrasound image may be a B-mode image or any suitable ultrasound image. The representation of the surgical needle 10 may be the color or grayscale shading of the shear wave speed in the velocity distribution map, the stiffness color or grayscale shading in the elasticity map, the color or grayscale shading in the spatial gradient map, a virtual representation of the needle 10 (e.g., a line, a generic image of a needle, etc.), or any suitable representation. For example, the position of the surgical needle 10 may be visible in the map based on the difference in velocity and/or elasticity between the needle 10 and surrounding tissue 1 and the characteristic shape of the needle 10. In an elasticity map, for example, the surgical needle 10 is a characteristic shape and has a stiffer elasticity than tissue 1 so that a representation of the needle 10 may appear in the elasticity map as a thin straight red or light gray/white line.

Aspects of the present invention provide a method 200 and system 100 for enhanced detection and visualization of a surgical needle 10 in ultrasound data by performing shear wave elasticity imaging. In accordance with various embodiments, the method 200 comprises inducing 204 a shear wave 5 in biological tissue 1 having a surgical needle 10 inserted therein. The method 200 comprises acquiring 206, by an ultrasound system 100, 104, shear wave ultrasound data at a high pulse repetition frequency from the biological tissue 1 having the surgical needle 10. The method 200 comprises processing 208, 210, by a processor 132, 140 of the ultrasound system 100, the shear wave ultrasound data to generate a map of the biological tissue 1 and the surgical needle 10. The method 200 comprises displaying 214, at a display system 134 of the ultrasound system 100, a representation of the surgical needle 10 overlaid on an ultrasound image.

In a representative embodiment, the inducing 204 the shear wave 5 comprises providing, by an ultrasound probe 104 of the ultrasound system 100, a high intensity ultrasound push pulse 105 into the biological tissue 1. In various embodiment, the inducing 204 the shear wave 5 comprises providing, by an external vibration device 20, an external push force 22 into the biological tissue 1. In certain embodiments, the high pulse repetition frequency is greater or equal to 1000 pulses/second.

In various embodiments, the processing 208, 210 the shear wave ultrasound data to generate the map comprises processing 208 the shear wave ultrasound data to determine a local distribution of shear wave speed through the biological tissue 1 and surgical needle 10. The processing 208, 210 the shear wave ultrasound data to generate the map comprises one or more of: converting 210 the location distribution of shear wave speed to a velocity distribution map of the biological tissue 1 and the surgical needle 10, converting 210 the local distribution of the shear wave speed to the elasticity map of the biological tissue 1 and the surgical needle 10, and applying 210 a spatial gradient filter to one or more of the velocity distribution map and the elasticity map to generate a spatial gradient map. In certain embodiments, the processing 208 the shear wave ultrasound data to determine a local distribution shear wave speed through the biological tissue 1 and the surgical needle 10 comprises computing the shear wave speed at locations in the shear wave ultrasound data by at least one of a direct inversion of a Helmholtz equation, and a time-of-flight measurement. In a representative embodiment, the converting 210 the local distribution of the shear wave speed to the elasticity map of the biological tissue 1 and the surgical needle 10 comprises computing elasticity based at least in part on one or more of Young's modulus and a shear modulus.

In certain embodiments, the map is a color-coded map and different colors in the color-coded map correspond with at least one of different velocities and different elasticities. In a representative embodiment, the representation of the surgical needle is at least one of a shear wave speed color in a velocity distribution map, an elasticity color in the elasticity map, and a virtual representation. In various embodiments, the method 200 comprises performing 212, by the processor 132, 140 of the ultrasound system 100, image segmentation of the surgical needle 10 in the map. In certain embodiments, the inducing 204 the shear wave in the biological tissue is initiated 202 based on an input at a user input module 130.

Various embodiments provide a system 100 comprising a shear wave inducement device 20, 104, an ultrasound processor 132, 140, and a display system 134. The shear wave inducement device 20, 104 may be configured to induce a shear wave 5 in biological tissue 1 having a surgical needle 10 inserted therein. The ultrasound processor 132, 140 may be configured to receive shear wave ultrasound data acquired at a high pulse repetition frequency from the biological tissue 1 having the surgical needle 10. The ultrasound processor 132, 140 may be configured to process the shear wave ultrasound data to generate a map of the biological tissue 1 and the surgical needle 10. The display system 134 may be configured to display a representation of the surgical needle 10 overlaid on an ultrasound image.

In certain embodiments, the shear wave inducement device is at least one of an ultrasound probe 104 configured to provide a high intensity ultrasound push pulse 105 into the biological tissue 1, and an external vibration device 20 configured to provide an external push force 22 into the biological tissue 1. In various embodiments, the ultrasound processor 132, 140 is configured to process the shear wave ultrasound data to generate the map by processing the shear wave ultrasound data to determine a local distribution of shear wave speed through the biological tissue 1 and surgical needle 10. The ultrasound processor 132, 140 is configured to process the shear wave ultrasound data to generate the map by one or more of converting the location distribution of the shear wave speed to a velocity distribution map of the biological tissue 1 and the surgical needle 10, converting the local distribution of the shear wave speed to the elasticity map of the biological tissue 1 and the surgical needle 10, and applying a spatial gradient filter to one or more of the velocity distribution map and the elasticity map to generate a spatial gradient map.

In a representative embodiment, the ultrasound processor 132, 140 is configured to perform image segmentation of the surgical needle 10 in the map. In certain embodiments, the system 100 comprises a user input module 130 configured to receive an input to initiate the inducement of the shear wave in the biological tissue 1 by the shear wave inducement device 20, 105. In various embodiments, the high pulse repetition frequency is greater or equal to 1000 pulses/second. In a representative embodiment, the map is a color-coded map, the different colors in the color-coded map correspond with at least one of different velocities and different elasticities, and the representation of the surgical needle 10 is at least one of a shear wave speed color in a velocity distribution map, an elasticity color in the elasticity map, and a virtual representation.

Certain embodiments provide a non-transitory computer readable medium having stored computer program comprises at least one code section that is executable by a machine for causing the machine to perform steps 200 disclosed herein. Exemplary steps 200 may comprise inducing 204 a shear wave 5 in biological tissue 1 having a surgical needle 10 inserted therein. The steps 200 may comprise acquiring 206 shear wave ultrasound data at a high pulse repetition frequency from the biological tissue 1 having the surgical needle 10. The steps 200 may comprise processing 208, 210 the shear wave ultrasound data to generate a map of the biological tissue 1 and the surgical needle 10. The steps 200 may comprise displaying 214 a representation of the surgical needle 10 overlaid on an ultrasound image.

In a representative embodiment, the processing 208, 210 the shear wave ultrasound data to generate the map comprises processing 208 the shear wave ultrasound data to determine a local distribution of shear wave speed through the biological tissue 1 and surgical needle 10. The processing 208, 210 the shear wave ultrasound data to generate the map comprises one or more of converting 210 the location distribution of the shear wave speed to a velocity distribution map of the biological tissue 1 and the surgical needle 10, converting 210 the local distribution of the shear wave speed to the elasticity map of the biological tissue 1 and the surgical needle 10, and applying a spatial gradient filter to one or more of the velocity distribution map and the elasticity map to generate a spatial gradient map.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code. As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and code (if any is necessary) to perform the function, regardless of whether performance of the function is disabled, or not enabled, by some user-configurable setting.

Other embodiments may provide a computer readable device and/or a non-transitory computer readable medium, and/or a machine readable device and/or a non-transitory machine readable medium, having stored thereon, a machine code and/or a computer program having at least one code section executable by a machine and/or a computer, thereby causing the machine and/or computer to perform the steps as described herein for enhanced detection and visualization of a surgical needle in ultrasound data by performing shear wave elasticity imaging.

Accordingly, various embodiments may be realized in hardware, software, or a combination of hardware and software. Certain embodiments may be realized in a centralized fashion in at least one computer system, or in a distributed fashion where different elements are spread across several interconnected computer systems. Any kind of computer system or other apparatus adapted for carrying out the methods described herein is suited. A typical combination of hardware and software may be a general-purpose computer system with a computer program that, when being loaded and executed, controls the computer system such that it carries out the methods described herein.

Certain embodiments may also be embedded in a computer program product, which comprises all the features enabling the implementation of the methods described herein, and which when loaded in a computer system is able to carry out these methods. Computer program in the present context means any expression, in any language, code or notation, of a set of instructions intended to cause a system having an information processing capability to perform a particular function either directly or after either or both of the following: a) conversion to another language, code or notation; b) reproduction in a different material form.

While the present invention has been described with reference to certain embodiments, it will be understood by those skilled in the art that various changes may be made and equivalents may be substituted without departing from the scope of the present invention. In addition, many modifications may be made to adapt a particular situation or material to the teachings of the present invention without departing from its scope. Therefore, it is intended that the present invention not be limited to the particular embodiment disclosed, but that the present invention will include all embodiments falling within the scope of the appended claims.

What is claimed is:

1. A method, comprising:
inducing a shear wave in biological tissue, wherein a surgical needle is inserted in the biological tissue;
acquiring, by an ultrasound system, shear wave ultrasound data at a high pulse repetition frequency from the biological tissue having the surgical needle;
processing, by a processor of the ultrasound system, the shear wave ultrasound data to generate a map of the biological tissue and the surgical needle; and
displaying, at a display system of the ultrasound system, a representation of the surgical needle overlaid on an ultrasound image.

2. The method according to claim 1, wherein the inducing the shear wave comprises providing, by an ultrasound probe of the ultrasound system, a high intensity ultrasound push pulse into the biological tissue.

3. The method according to claim 1, wherein the inducing the shear wave comprises providing, by an external vibration device, an external push force into the biological tissue.

4. The method according to claim 1, wherein the high pulse repetition frequency is greater or equal to 1000 pulses/second.

5. The method according to claim 1, wherein the processing the shear wave ultrasound data to generate the map comprises:
processing the shear wave ultrasound data to determine a local distribution of shear wave speed through the biological tissue and surgical needle; and
one or more of:
converting the location distribution of shear wave speed to a velocity distribution map of the biological tissue and the surgical needle,
converting the local distribution of the shear wave speed to an elasticity map of the biological tissue and the surgical needle, and
applying a spatial gradient filter to one or more of the velocity distribution map and the elasticity map to generate a spatial gradient map.

6. The method according to claim 5, wherein the processing the shear wave ultrasound data to determine a local distribution shear wave speed through the biological tissue and the surgical needle comprises computing the shear wave speed at locations in the shear wave ultrasound data by at least one of:
a direct inversion of a Helmholtz equation, and
a time-of-flight measurement.

7. The method according to claim 5, wherein the converting the local distribution of the shear wave speed to the elasticity map of the biological tissue and the surgical needle comprises computing elasticity based at least in part on one or more of:
Young's modulus, and
a shear modulus.

8. The method according to claim 1, wherein the map is a color-coded map, and wherein different colors in the color-coded map correspond with at least one of different velocities and different elasticities.

9. The method according to claim 1, wherein the representation of the surgical needle is at least one of:
a shear wave speed color in a velocity distribution map,
an elasticity color in the elasticity map, and
a virtual representation.

10. The method according to claim 1, comprising performing, by the processor of the ultrasound system, image segmentation of the surgical needle in the map.

11. The method according to claim 1, wherein the inducing the shear wave in the biological tissue is initiated based on an input at a user input module.

12. A system, comprising:
a shear wave inducement device configured to induce a shear wave in biological tissue, wherein a surgical needle is inserted in the biological tissue;
an ultrasound processor configured to:
receive shear wave ultrasound data acquired at a high pulse repetition frequency from the biological tissue having the surgical needle; and
process the shear wave ultrasound data to generate a map of the biological tissue and the surgical needle; and
a display system configured to display a representation of the surgical needle overlaid on an ultrasound image.

13. The system according to claim 12, wherein the shear wave inducement device is at least one of:
an ultrasound probe configured to provide a high intensity ultrasound push pulse into the biological tissue, and
an external vibration device configured to provide an external push force into the biological tissue.

14. The system according to claim 12, wherein the ultrasound processor is configured to process the shear wave ultrasound data to generate the map by:
processing the shear wave ultrasound data to determine a local distribution of shear wave speed through the biological tissue and surgical needle; and
one or more of:
converting the location distribution of shear wave speed to a velocity distribution map of the biological tissue and the surgical needle,
converting the local distribution of the shear wave speed to an elasticity map of the biological tissue and the surgical needle, and
applying a spatial gradient filter to one or more of the velocity distribution map and the elasticity map to generate a spatial gradient map.

15. The system according to claim 12, wherein the ultrasound processor is configured to perform image segmentation of the surgical needle in the map.

16. The system according to claim 12, comprising a user input module configured to receive an input to initiate the inducement of the shear wave in the biological tissue by the shear wave inducement device.

17. The system according to claim 12, wherein the high pulse repetition frequency is greater or equal to 1000 pulses/second.

18. The system according to claim 12, wherein:
the map is a color-coded map;
different colors in the color-coded map correspond with at least one of different velocities and different elasticities; and
the representation of the surgical needle is at least one of:
a shear wave speed color in a velocity distribution map,
an elasticity color in the elasticity map, and
a virtual representation.

19. A non-transitory computer readable medium having stored thereon, a computer program having at least one code section, the at least one code section being executable by a machine for causing the machine to perform steps comprising:
inducing a shear wave in biological tissue, wherein a surgical needle is inserted in the biological tissue;
acquiring shear wave ultrasound data at a high pulse repetition frequency from the biological tissue having the surgical needle;
processing the shear wave ultrasound data to generate a map of the biological tissue and the surgical needle; and
displaying a representation of the surgical needle overlaid on an ultrasound image.

20. The non-transitory computer readable medium according to claim 19, wherein the processing the shear wave ultrasound data to generate the map comprises:
processing the shear wave ultrasound data to determine a local distribution of shear wave speed through the biological tissue and surgical needle; and
one or more of:
converting the location distribution of shear wave speed to a velocity distribution map of the biological tissue and the surgical needle,
converting the local distribution of the shear wave speed to an elasticity map of the biological tissue and the surgical needle, and
applying a spatial gradient filter to one or more of the velocity distribution map and the elasticity map to generate a spatial gradient map.
